(19) United States
(12) Patent Application Publication
(10) Pub. No.: US 2012/0087903 A1
(43) Pub. Date: Apr. 12, 2012

(54) Hair growth agent having a platelet dry powder

(75) Inventors: Chin I Lin, Taipei (TW); Han-Lei Wan, Taipei (TW)

(73) Assignee: Central Medical Technologies Inc., Taipei (TW)

(21) Appl. No.: 13/200,650

(22) Filed: Sep. 28, 2011

(30) Foreign Application Priority Data
Oct. 6, 2010 (TW) 99134114

(51) Int. Cl. A61K 35/14; A61P 17/14

(52) U.S. Cl. 424/93.72

(57) ABSTRACT

A hair growth agent having an effective dose of a platelet dry powder and a pharmaceutically acceptable solvent and/or excipient, wherein the effective dose refers to the presence of at least 1,000 platelets in every milligram of the hair growth agent.

### FIELD OF THE INVENTION

The present invention relates to a hair growth agent and usage thereof, and more particularly to a hair growth agent using compositions of platelet dry powder for promoting hair growth and usage thereof.

### BACKGROUND OF THE INVENTION

Hair loss is a nagging problem many people face today, which subsequently makes developing methods of hair growth an important goal. For instance, in the published Taiwan patent application 200841886: a dual-agent of hair growth comprising active ingredients of 0.02%-2% of nitrite, and 0.5%-4% of acidic agents was disclosed; or in the published Taiwan patent application 200413014: a hair growth agent having abnormal expression of growth/transcription factors was disclosed. Moreover, in the announced Taiwan patents 128728, 191212, 260665, 474821, 490307, 501931, 592719, and in the published Taiwan patent applications 200509957, 200612904, and 200825399, all of which had disclosed hair growth agents using small-molecule chemicals as active ingredients thereof. In the announced Taiwan patent 518220, a hair growth agent formula using the FKBP ligands as active ingredients was disclosed, whereas in the announced Taiwan patent 531419, a hair growth agent formula having serine proteinases as active ingredients was disclosed. In addition, the published Taiwan patent application 200409650 had disclosed a hair growth agent formula using vitamins and/or co-enzymes as active ingredients; the published Taiwan patent applications 200509972, 200529876, and 200942251 had disclosed hair growth agent formulas including plant extracts as active ingredients, and the published Taiwan patent application 200815038 had disclosed a hair growth agent formula having estrogen as the active ingredient. However, a hair growth agent using a dry powder of platelets as the active ingredient has not been developed until now. In the US patents, the only patent that had disclosed a hair growth agent utilizing platelets-related substances as the active ingredient is the published patent application 20050049268, and the patent disclosed a hair growth agent using PDGF receptor tyrosine kinases inhibitor as the active ingredient. The present invention is the first to have disclosed a hair growth agent using a platelet dry powder as the active ingredient, which has been applied to promote hair growth in humans, and had achieved successful results subsequently.

### SUMMARY OF THE INVENTION

The present invention provides a new usage of applying a platelet dry powder to promote hair growth.

The present invention provides a hair growth agent using a platelet dry powder as an active ingredient thereof.

The present invention provides a method based on a formulation of platelet dry powder for promoting hair growth.

The hair growth agent of the present invention is suitable for promoting hair growth in animals, and especially body hair in humans such as hair and eyebrows.

The hair growth agent of the present invention includes an effective dose of platelets for hair growth and a pharmaceutically acceptable solvent and/or excipient, wherein the effective dose refers to the presence of at least 1000 platelets in every milligram of hair growth agent. In a preferred embodiment of the invention, there are 1000 to 15000 platelets present in every milligram of hair growth agent.

The source of said platelets of the hair growth agent of the present invention is a platelet dry powder. Said platelet dry powder is prepared from blood or blood preparations like platelet-rich plasma (PRP) by using specific methods, and comprises intact platelets. The methods for preparing the platelet dry powder may be any of the previously known methods for preparing platelet dry powders, as can be referred to in the announced Taiwan patents 1300806 and 1270375; the published Taiwan patent applications 201004659 and 200526680, and the announced US patents 7659052, 7202020, 7169606, 6060233, 5736313, and 5589462. Moreover, the preparation of the platelet dry powder described in this invention is not limited to the methods disclosed in the aforesaid cited references. Due to the application of different preparation methods, said platelet dry powder may comprise low amounts of anticoagulants and protectants, such as the platelet dry powder prepared according to the method disclosed in the announced Taiwan patent TW-1270375, which includes low amounts of the anticoagulant acid citrate dextrose, cryoprecipitates, and thrombin, but since said components do not actually affect the performance of the platelet dry powder, it is not necessary to eliminate them therefrom.

Said hair growth agent may be any of the previously known forms of drugs, such as solutions, suspensions, ointments, powders, and pills; is preferably in the form of solutions, ointments, or transdermal patches, and is more preferably in the form of sprays or ointments. In addition, the powders and pills may be prepared into other forms suitable for use in actual applications.

Said pharmaceutically acceptable solvent refers to any solvents that can be ingested or applied externally by humans or animals, such as alcohol-water co-solvents, water, and saline, and is preferably water or saline. In addition, it is critical to ensure the amount of solvent added could maintain the effective dose of platelets.

The pharmaceutically acceptable excipient is the previously known excipients, and applications thereof are determined according to the dosage form of the hair growth agent.

Said effective dose of platelet dry powder refers to the presence of at least 1000 platelets in every milligram of the hair growth agent; is preferably to be the presence of at least 2000 platelets therein, and is more preferably to be the presence of at least 5000 platelets therein.

Said hair growth agent may be added with any effective ingredients that convey positive effects (such as anti-inflammatory ingredients, analgesic ingredients, nutritious ingredients, and/or ingredients promoting absorption), or any ingredients that do not have negative effects or side effects (such as spices).

Said platelet dry powder may be heterologous, homologous, or autologous platelet dry powders. Considering the users' possible concerns, the platelet dry powder is preferably a homologous or an autologous platelet dry powder, and is more preferably an autologous platelet dry powder.

Generally speaking, the platelet-derivatives growth factors (PDGF) of platelets are approximately 40-200 pg/mL, as can be referred to in Vogt., et al., Determination of endogenous growth factors in human wound healing. Wound Repair Regeneration, 2004, 12(4): p. 485-492. The effectiveness of the hair growth agent of the present invention may be related to the long-term effectiveness of PDGF of the platelet dry powder, but the relationship thereof still requires further researches to confirm.

A method for promoting hair growth according to the present invention comprises:

a cleaning step to make a body area awaiting the promotion of hair growth suitable for further treatments; and

a spraying and spreading step, which sprays or spreads a hair growth agent having an effective dose of platelets over said cleaned body area, wherein the effective dose refers to the presence of at least 1000 platelets in every milligram of hair growth agent.

Preferably, the body area is on scalp.

Said cleaning step refers to steps like rinsing, disinfecting, or combing hair away from the body area awaiting the promotion of hair growth into a condition suitable for further treatments. The necessity of rinsing the body area awaiting the promotion of hair growth generally depends on the cleanness of the body area, and the body area is preferably rinsed in advance or immediately before treatments. Though disinfecting is not absolutely needed, it is preferable to have the body area disinfected thoroughly. The need for combing hair from the body area depends on actual requirements, and if the body area awaiting the promotion of hair growth does not actually possess any hair, or is almost without any hair, it does not require combing (yet if the body areas are still surrounded by hair, it is ideal to comb the surrounding hair away from the body areas). If the body areas awaiting the promotion of hair growth actually possessed hair, it would be ideal to comb the hair into a condition suitable for further treatments.

Said spraying and spreading step refers to spraying or spreading the hair growth agent having an effective dose of platelets over said cleaned body area according to the form of the hair growth agent. For example, if the hair growth agent is a solution, it could be evenly sprayed on the body area awaiting the promotion of hair growth, and if the hair growth agent is an ointment, it could be evenly spread on the body area awaiting the promotion of hair growth.

Said method for promoting hair growth is suitable for use on animals having hair, especially humans, and also including pets.

In order to enhance the effects of hair growth promotion, a further absorption-enhancing step may be added before, during, or after the cleaning step, and is preferably added after disinfecting and before combing.

Said absorption-enhancing step may be applied in any forms that facilitates the absorption of a hair growth agent, such as massaging or laser massaging to promote blood circulation to hair follicles, electromagnetic wave introduction, and micro-needle penetration; is preferably electromagnetic wave introduction or micro-needle penetration, and is more preferably micro-needle penetration.

### BRIEF DESCRIPTION OF DRAWINGS

FIG. 1a is a photograph showing the hair condition of the patient in Embodiment 1 before treatment, while FIGS. 1b, 1c, and 1d are photographs showing the hair condition of the patient after treatment.

FIG. 2a is a photograph showing the hair condition of the patient in Embodiment 2 before treatment, while FIGS. 2b and 2c are photographs showing the hair condition of the patient after treatment.

FIG. 3a is a photograph showing the hair condition of the patient in Embodiment 3 before treatment, while FIGS. 3b and 3c are photographs showing the hair condition of the patient after treatment.

FIG. 4a is a photograph showing the hair condition of the patient in Embodiment 4 before treatment, while FIGS. 4b and 4c are photographs showing the hair condition of the patient after treatment.

FIG. 5a is a photograph showing the hair condition of the patient in Embodiment 5 before treatment, while FIG. 5b is a photograph showing the hair condition of the patient after treatment.

FIG. 6a is a photograph showing the hair condition of the patient in Embodiment 6 before treatment, while FIG. 6b is a photograph showing the hair condition of the patient after treatment.

FIG. 7a is a photograph showing the hair condition of the patient in Embodiment 7 before treatment, while FIG. 7b is a photograph showing the hair condition of the patient after treatment.

FIG. 8a is a photograph showing the hair condition of the patient in Embodiment 8 before treatment, while FIG. 8b is a photograph showing the hair condition of the patient after treatment.

FIG. 9a is a photograph showing the hair condition of the patient in Embodiment 9 before treatment, while FIG. 9b is a photograph showing the hair condition of the patient after treatment.

### DETAILED DESCRIPTION OF PREFERRED EMBODIMENTS

The preferred embodiments are described hereafter in order to further elucidate the techniques of the invention:

### Testing Example:

The method disclosed in the announced Taiwan patent TW-1270375 was used to prepare a platelet dry powder, in which 1.0 g of platelet dry powder was obtained and added to saline to make up 5.0 mL of solution, then the PDGF titer of the solution was analyzed by using a spectrophotometer (U.S. Bio-Tek Instruments, Inc., Model μ-Quant) immediately after preparation (0 week), after 1 week, after 2 weeks, after 3 weeks, after 4 weeks, after 5 weeks, and after 6 weeks. Results from the analyses are shown in Table 1.

TABLE 1
Changes of PDGF titer in solutions prepared
from the platelet dry powder.

| Weeks | 0 | 1 | 2 | 3 | 4 | 5 | 6 |
|---|---|---|---|---|---|---|---|
| PDGF | 2930 | 2795 | 3012 | 2854 | 2822 | 2776 | 2785 |
| (pg/mL) | | | | | | | |

The experimental figures indicated the PDGF titer of the platelet dry powder is very stable.

### Embodiment 1

The method disclosed in the announced patent TW-1270375 was used to prepare an autologous platelet dry powder from autologous blood in advance. The platelet dry powder was added to reverse osmosis (RO) water, in order to prepare a solution having 1000 platelets/μL, and then held in a spray bottle. The number of platelets in the solution was measured by using a Hematology Analyzer (Manufacturer: Sysmex, Model: KX-21).

A patient was subjected to a treatment for promoting hair growth once a week, which involved initially disinfecting a body area awaiting the promotion of hair growth, then rolling a micro-roller (purchased from the Korean company Daesung Medical Co. Ltd., Model Mi-Roll-1) over the disinfected body area (micro-needle penetration), and finally evenly spraying the hair growth agent (solution) on the disinfected body area. In addition, the body area awaiting the promotion of hair growth was subjected to spraying of the hair growth agent evenly after washing hair everyday between every two treatments.

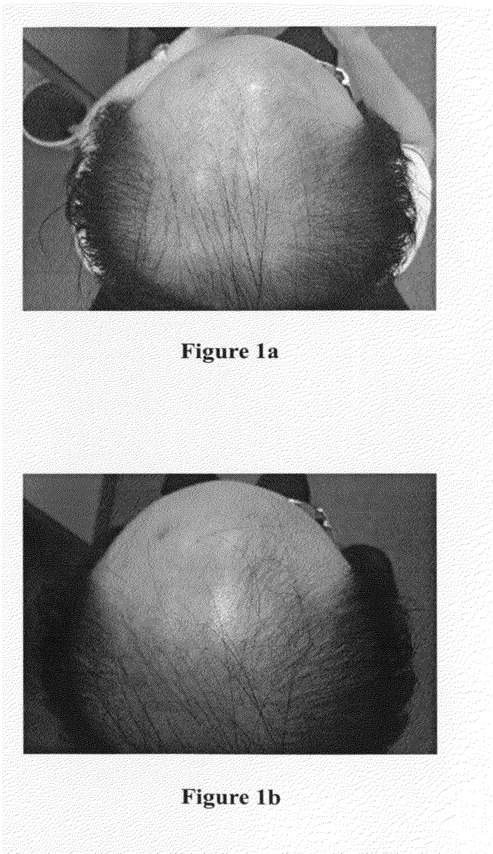

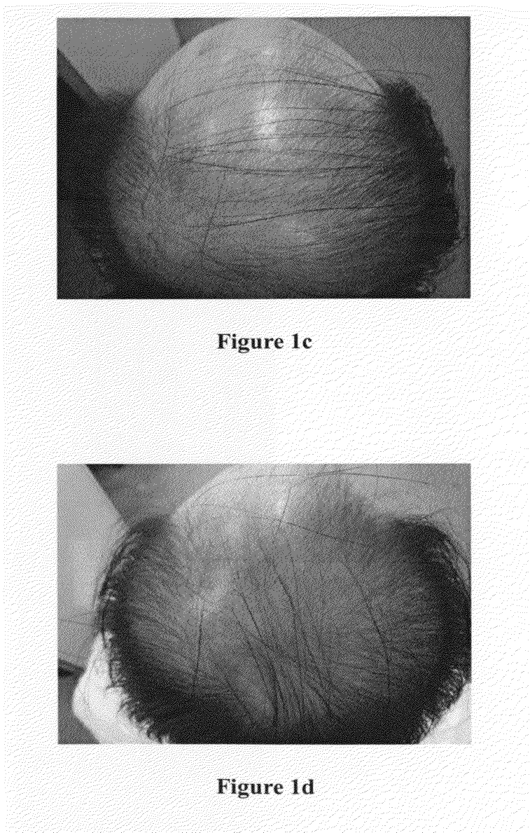

FIGS. 1a and 1b show the hair on the body area awaiting the promotion of hair growth before the treatments and 3 weeks after the treatments; though there appeared to be hair growth, it was insignificant.

Similar to the treatment described above, but the concentration of the hair growth agent was increased to 2000 platelets/μL, and the evidence of hair growth in the fourth week was more significant than the previous three weeks, as could be seen in FIG. 1c.

Similar to the aforesaid treatment, but the concentration of the hair growth agent was increased to 5000 platelets/μL, and the evidence of hair growth in the eighth week was even more significant than that in the fourth week; with abundant growth of new hair, as shown in FIG. 1d.

### Embodiment 2

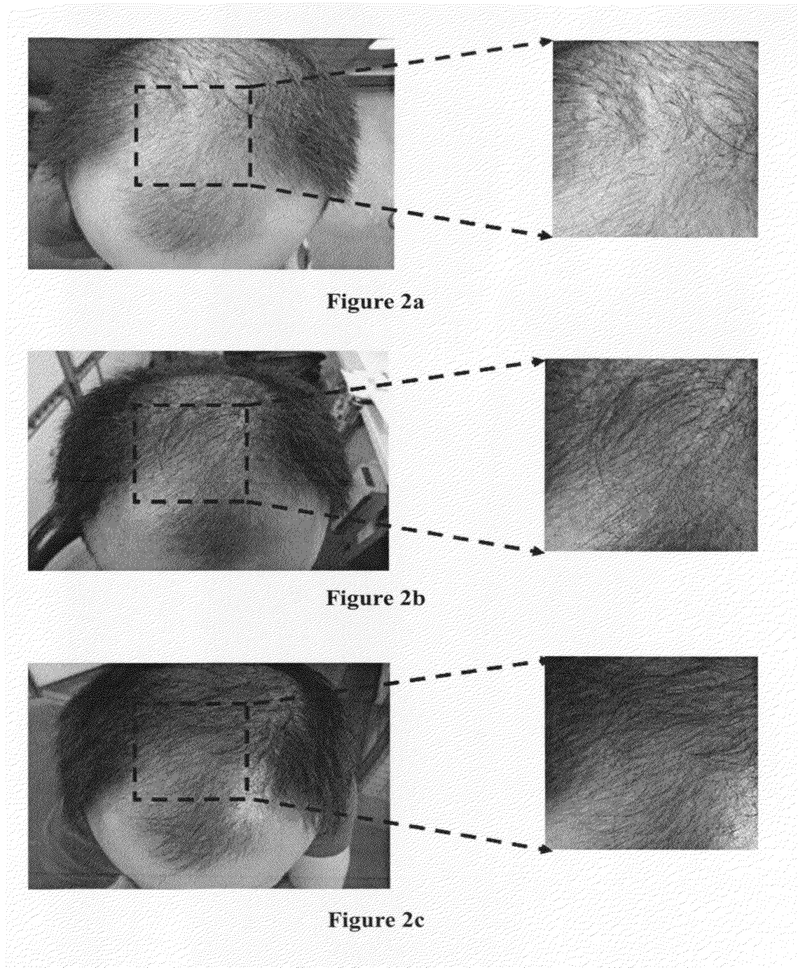

Similar to Embodiment 1, but the platelet dry powder was a homologous platelet dry powder prepared from the blood in a blood bank, and the concentration of the hair growth agent (platelet number/μL of solution) was 5000. FIGS. 2a, 2b, and 2c show the hair conditions before the treatments, 5 weeks after the treatments, and 7 weeks after the treatments for the body area awaiting the promotion of hair growth, and the pictures on the right of each of the photographs are the magnified pictures of the highlighted parts on the photographs. The results show that there is significant growth of new hair (black hair), and the old hair became thickened.

### Embodiment 3

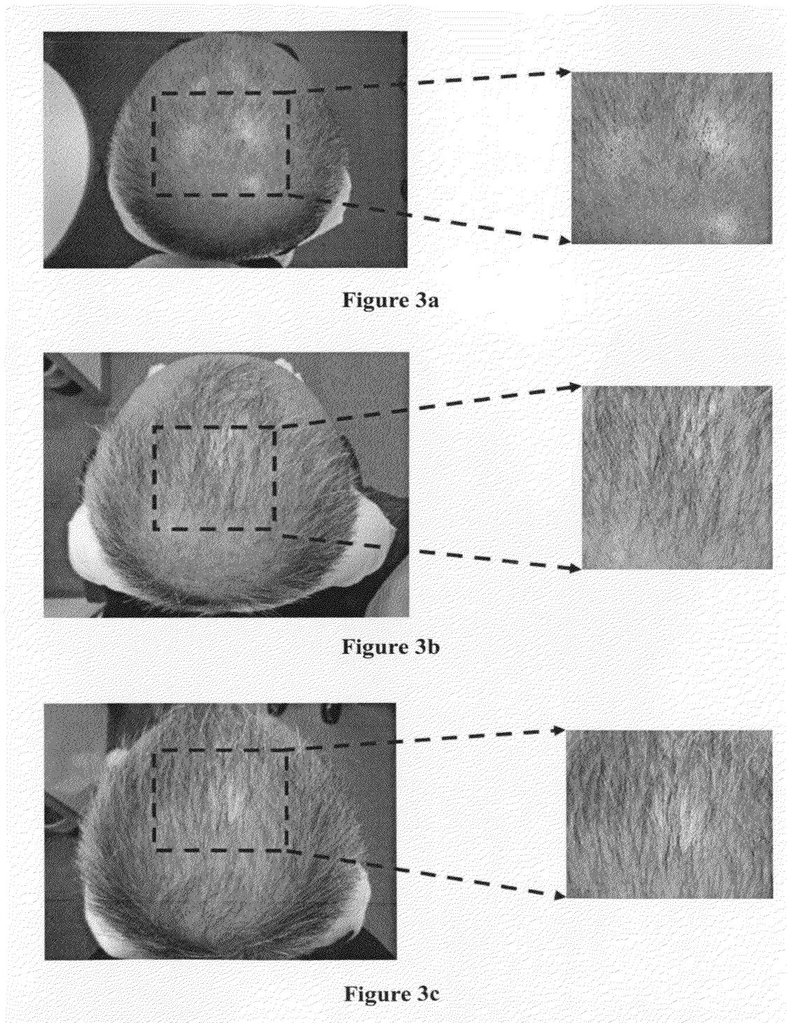

Similar to Embodiment 2, FIGS. 3a, 3b, and 3c show the hair conditions before the treatments, as well as 1 week and 3 weeks after the treatments for the body area awaiting the promotion of hair growth, and the pictures on the right of each of the photographs are the magnified pictures of the highlighted parts on the photographs. The results show that there is significant growth of new hair (black hair), and the old hair became thickened.

### Embodiment 4

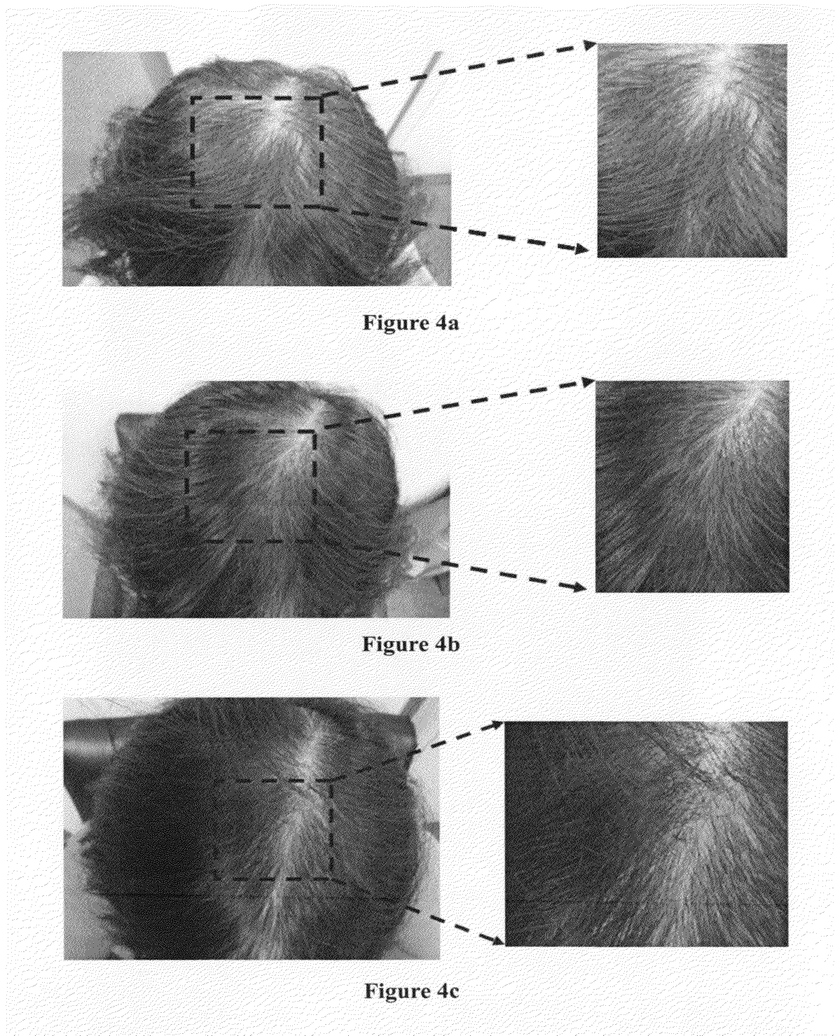

Similar to Embodiment 2, FIGS. 4a, 4b, and 4c show the hair conditions before the treatments, as well as 5 weeks and 7 weeks after the treatments for the body area awaiting the promotion of hair growth, and the pictures on the right of each of the photographs are the magnified pictures of the highlighted parts on the photographs. The results show that there is significant growth of new hair (black hair), and the old hair became thickened.

### Embodiment 5

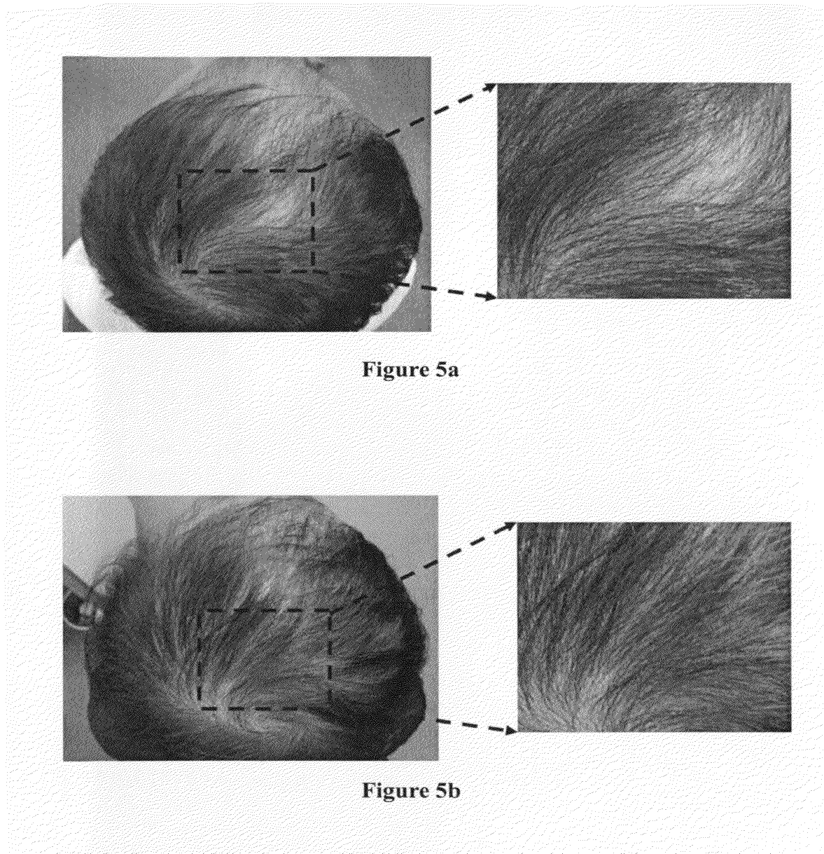

Similar to Embodiment 2, FIGS. 5a and 5b show the hair conditions before the treatments and 4 weeks after the treatments for the body area awaiting the promotion of hair growth, and the pictures on the right of each of the photographs are the magnified pictures of the highlighted parts on the photographs. The results indicate that there is significant growth of new hair (black hair), and the old hair became thickened.

### Embodiment 6

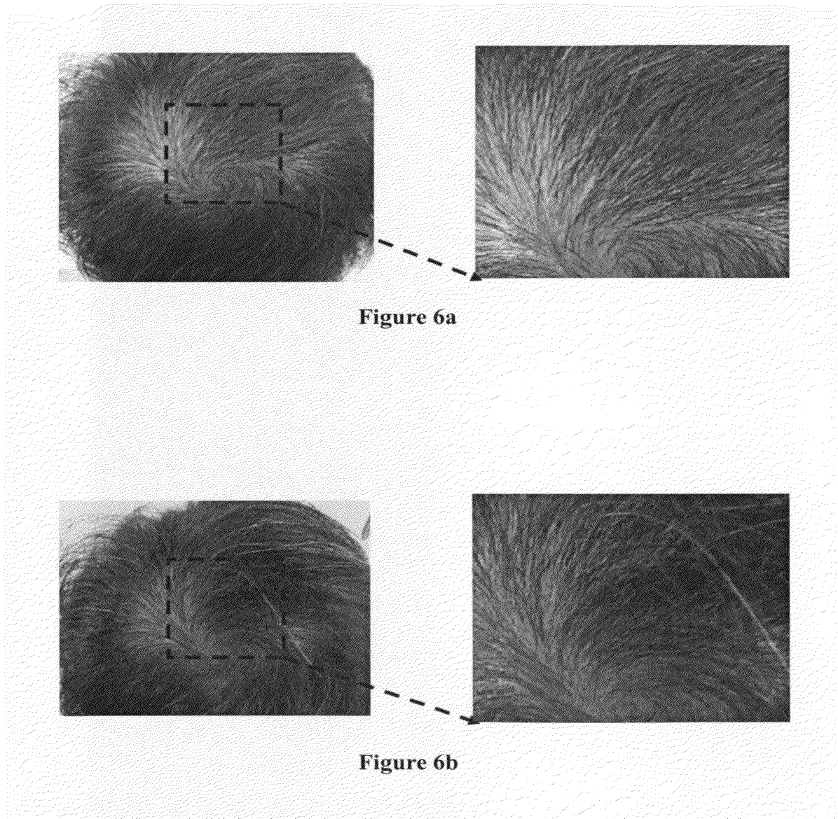

Similar to Embodiment 2, FIGS. 6a and 6b show the hair conditions before the treatments and 5 weeks after the treatments for the body area awaiting the promotion of hair growth, and the pictures on the right of each of the photographs are the magnified pictures of the highlighted parts on the photographs. The results indicate that there is significant growth of new hair (black hair), and the old hair became thickened.

### Embodiment 7

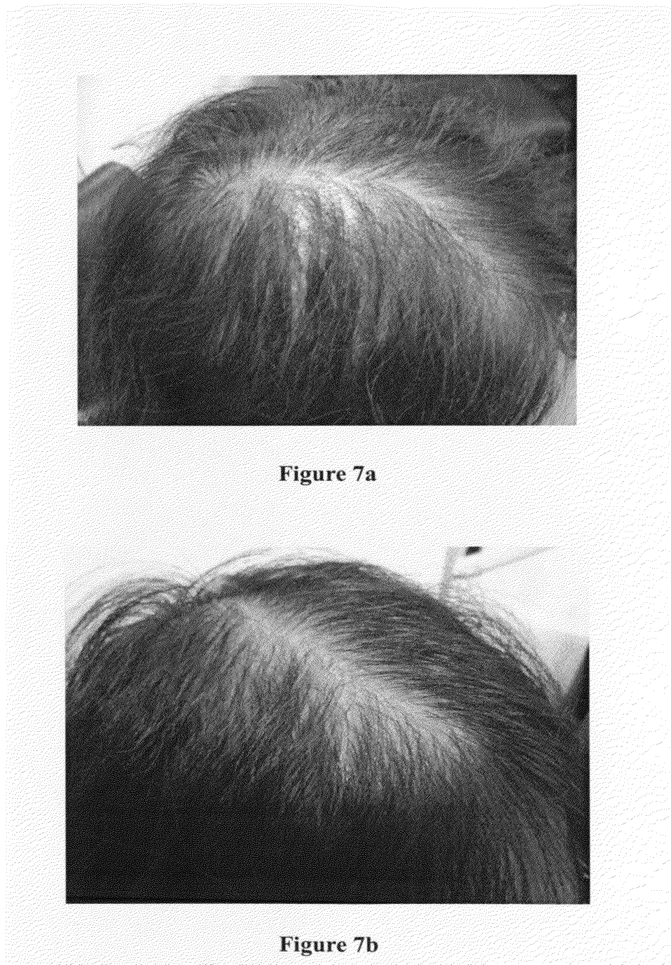

Similar to Embodiment 2, FIGS. 7a and 7b show the hair conditions before the treatments and 2 weeks after the treatments for the body area awaiting the promotion of hair growth, and the results indicate that there are significant growth of new hair, and the old hair became thickened.

### Embodiment 8

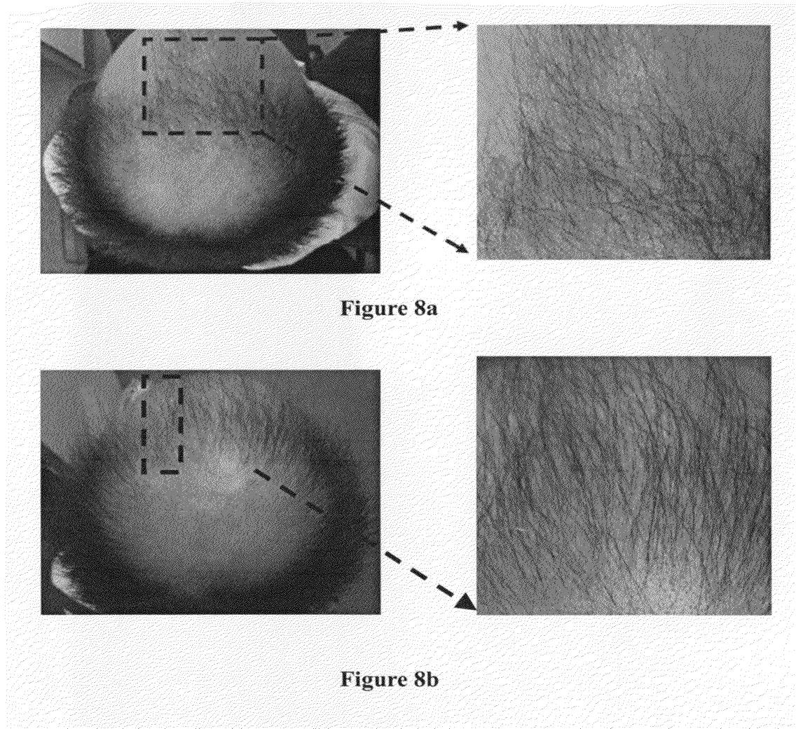

Similar to Embodiment 2, FIGS. 8a and 8b show the hair conditions before the treatments and 6 weeks after the treatments for the body area awaiting the promotion of hair growth, and the pictures on the right of each of the photographs are the magnified pictures of the highlighted parts on the photographs. The results show that there is significant growth of new hair (black hair), and the old hair became thickened.

### Embodiment 9

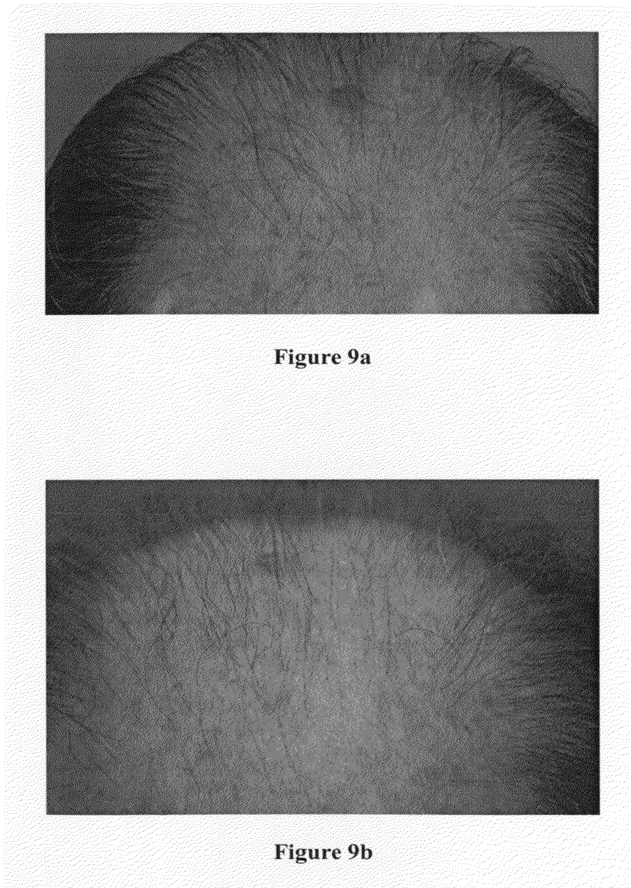

Similar to Embodiment 2, FIGS. 9a and 9b show the hair conditions before the treatments and 6 weeks after the treatments for the body area awaiting the promotion of hair growth, and the results show that there is significant growth of new hair, and the old hair became thickened.

### Embodiments 10-24

Similar to Embodiments 2-9, the platelet dry powder (abbreviated as PLT hereafter) was prepared by using the same method. The concentration of the hair growth agent (platelet number/μL of solution) and the effects of the treatments after 4 weeks and 8 weeks are presented in Table 2. In the table, the homologous PLT was prepared from the blood of a blood bank; the heterologous PLT was prepared from bovine blood, and the effects of the treatments at 4 and 8 weeks were recorded.

TABLE 2
Conditions and results of the treatments for Embodiments 10-24

| | | | Effect of 4-week | Effect of 8-week |
|---|---|---|---|---|
| Embodiment | PLT | Conc. | treatment*-*** | treatment*-*** |
| 10 | Autologous | 1000 | Insignificant | * |
| 11 | Autologous | 2000 | * | ** |
| 12 | Autologous | 5000 | *** | *** |
| 13 | Autologous | 10000 | *** | *** |
| 14 | Autologous | 15000 | *** | *** |
| 15 | Homologous | 1000 | Insignificant | * |
| 16 | Homologous | 2000 | * | ** |
| 17 | Homologous | 5000 | *** | *** |
| 18 | Homologous | 10000 | *** | *** |
| 19 | Homologous | 15000 | *** | *** |
| 20 | Heterologous | 1000 | * | ** |
| 21 | Heterologous | 2000 | ** | *** |
| 22 | Heterologous | 5000 | *** | *** |
| 23 | Heterologous | 10000 | *** | *** |
| 24 | Heterologous | 15000 | *** | *** |

*Slight growth of new hair
**Apparent growth of new hair
***Significant growth of new hair, thickening of hair

Results from Table 2 Shows:

In regard to the effects of the treatments, the heterologous PLT appeared to outperform the homologous PLT and the autologous PLT. However, the patients might still prefer to use homologous PLT or autologous PLT.

What is claimed is:

1. A hair growth agent comprising an effective dose of platelets for hair growth and a pharmaceutically acceptable solvent and/or excipient, wherein the effective dose refers to the presence of at least 1,000 platelets in every milligram of the hair growth agent.

2. The hair growth agent of claim 1 comprising an effective dose of platelets and a pharmaceutically acceptable solvent.

3. The hair growth agent of claim 2, wherein the solvent is water or saline.

4. The hair growth agent of claim 1, wherein the platelets are autologous platelets.

5. The hair growth agent of claim 1, wherein the effective dose refers to the presence of at least 2000 platelets in every milligram of the hair growth agent.

6. The hair growth agent of claim 2, wherein the effective dose refers to the presence of at least 2000 platelets in every milligram of the hair growth agent.

7. The hair growth agent of claim 3, wherein the effective dose refers to the presence of at least 2000 platelets in every milligram of the hair growth agent.

8. The hair growth agent of claim 4, wherein the effective dose refers to the presence of at least 2000 platelets in every milligram of the hair growth agent.

9. The hair growth agent of claim 5, wherein the effective dose refers to the presence of at least 5000 platelets in every milligram of hair growth agent.

10. The hair growth agent of claim 6, wherein the effective dose refers to the presence of at least 5000 platelets in every milligram of hair growth agent.

11. The hair growth agent of claim 7, wherein the effective dose refers to the presence of at least 5000 platelets in every milligram of hair growth agent.

12. The hair growth agent of claim 8, wherein the effective dose refers to the presence of at least 5000 platelets in every milligram of hair growth agent.

13. The hair growth agent of claim 1, wherein a source of said platelets is a platelet dry powder.

14. A method for promoting hair growth comprising:
a spraying and spreading step, which sprays or spreads a hair growth agent comprising an effective dose of platelets for hair growth over a body area where hair grows, wherein the effective dose refers to the presence of at least 1000 platelets in every milligram of hair growth agent.

15. The method of claim 14 further comprising a cleaning step before said spraying and spreading step, which involves combing hair away from, rinsing, or disinfecting the body area awaiting the promotion of hair growth.

16. The method of claim 15 further comprising an absorption-enhancing step, and said absorption-enhancing step is executed before, during, or after the cleaning step.

17. The method of claim 16, wherein said absorption-enhancing step is executed after the cleaning step.

18. The method of claim 15, wherein said hair growth agent is a solution having an effective dose of platelets, and the spraying and spreading step sprays the solution of the platelets over the body area being cleaned.

19. The method of claim 18, wherein the solution contains water or saline as a solvent thereof.

20. The method of claim 14, wherein the platelets are autologous platelets.

21. The method of claim 14, wherein the effective dose refers to the presence of at least 2000 platelets in every milligram of hair growth agent.

22. The method of claim 15, wherein the effective dose refers to the presence of at least 2000 platelets in every milligram of hair growth agent.

23. The method of claim 16, wherein the effective dose refers to the presence of at least 2000 platelets in every milligram of hair growth agent.

24. The method of claim 17, wherein the effective dose refers to the presence of at least 2000 platelets in every milligram of hair growth agent.

25. The method of claim 18, wherein the effective dose refers to the presence of at least 2000 platelets in every milligram of hair growth agent.

26. The method of claim 19, wherein the effective dose refers to the presence of at least 2000 platelets in every milligram of hair growth agent.

27. The method of claim 20, wherein the effective dose refers to the presence of at least 2000 platelets in every milligram of hair growth agent.

28. The method of claim 21, wherein the effective dose is the presence of at least 5000 platelets in every milligram of hair growth agent.

29. The method of claim 22, wherein the effective dose is the presence of at least 5000 platelets in every milligram of hair growth agent.

30. The method of claim 23, wherein the effective dose is the presence of at least 5000 platelets in every milligram of hair growth agent.

31. The method of claim 24, wherein the effective dose is the presence of at least 5000 platelets in every milligram of hair growth agent.

32. The method of claim 25, wherein the effective dose is the presence of at least 5000 platelets in every milligram of hair growth agent.

33. The method of claim 26, wherein the effective dose is the presence of at least 5000 platelets in every milligram of hair growth agent.

34. The method of claim 27, wherein the effective dose is the presence of at least 5000 platelets in every milligram of hair growth agent.

35. The method of claim 24, wherein the body area is on scalp.

36. The method of claim 14, wherein a source of said platelets is a platelet dry powder.
